(19) United States
(12) Patent Application Publication
(10) Pub. No.: US 2005/0048492 A1
(43) Pub. Date: Mar. 3, 2005

(54) Methods of identifying genetic risk for and evaluating treatment of alzheimer's disease by determining cyp46 genotype

(76) Inventors: Andreas Papassotiropoulos, Zurich (CH); Johannes R. Streffer, Zurich (CH); Roger M. Nitsch, Zollikon (CH); Christoph Hock, Zurich (CH)

Correspondence Address: JACOBSON HOLMAN PLLC, 400 SEVENTH STREET N.W., SUITE 600, WASHINGTON, DC, 20004, US

(21) Appl. No.: 10/495,261

(22) PCT Filed: Mar. 1, 2002
(86) PCT No.: PCT/EP02/02211

Related U.S. Application Data
(60) Provisional application No. 60/331,174, filed on Nov. 9, 2001.

(30) Foreign Application Priority Data
Nov. 9, 2001 (EP) 01126773.9

(51) Int. Cl. C12Q 1/68

(52) U.S. Cl. 435/6

(57) ABSTRACT

Based on the unexpected identification of a CYP46 gene polymorphism as a novel genetic risk factor that links cholesterol metabolism to Alzheimer's disease, the present invention provides a method of diagnosing or prognosticating Alzheimer's disease, or determining the propensity or predisposition of a subject to develop Alzheimer's disease. The method comprises detecting the presence or absence of a variation in the CYP46 gene which encodes the enzyme cholesterol 24-hydroxylase.

Neurodegenerative diseases, in particular Alzheimer's disease, have a severely debilitating impact on a patient's life. Furthermore, these diseases constitute an enormous health, social, and economic burden. Alzheimer's disease is the most common age-related neurodegenerative condition affecting about 10% of the population over 65 years of age and up to 45% over age 85 (for a recent review see Vickers et al., Progress in Neurobiology 2000, 60:139-165; the contents of all publications, patents and patent applications referred to and cited in the present invention shall be incorporated by reference in their entirety). Presently, this amounts to an estimated 12 million cases in the US, Europe, and Japan. This situation will inevitably worsen with the demographic increase in the number of old people (“aging of the baby boomers”) in developed countries. The neuropathological hallmarks that occur in the brain of individuals suffering from Alzheimer's disease are senile plaques, composed of amyloid-b protein, and profound cytoskeletal changes coinciding with the appearance of abnormal filamentous structures and the formation of neurofibrillary tangles. AD is a progressive disease that is associated with early deficits in memory formation and ultimately leads to the complete erosion of higher cognitive function. Alzheimer's disease is genetically complex. The risk for the development of AD is determined by variations of genes involved in major pathophysiological pathways of this disorder. A considerable part of this risk is attributed to the inheritance of the e4 allele of the apolipoprotein E gene (APOE*4). However, several additional genes and genetic interactions add to the overall genetically determined susceptibility for the development of AD.

Genes coding for proteins involved in central disease-related pathways are of particular interest in the genetics of AD. The overproduction and aggregation of the b-amyloid peptide (Ab) in the hippocampus and the medial lobe (MTL) is a crucial step in the pathogenesis of AD. Thus, genes implicated in mechanisms leading to Ab accumulation are promising candidates in the search for susceptibility genes of AD.

Brain deposition of b-amyloid peptide (Ab) is a crucial step in the pathogenesis of AD (Hardy J A, et al., Science, 256:184-5, 1992). It can cause the formation of neurofibrillary tangles within neurons (Götz J, et al., Science, 293:1491-5, 2001; Lewis J, et al., Science, 293:1487-91, 2001). The concentration of the amyloid peptide Ab42 may be used as a surrogate, quantitative trait to identify genetic loci for AD (Ertekin-Taner N, et al., Science, 290:2303-4, 2000). Thus, genes implicated in the regulation of Ab formation and its degradation are candidate susceptibility genes for AD. Recent observations link brain levels of cholesterol to the regulation of the endoproteolytic processing of APP, and to Ab production (Simons M, et al., Neurology, 57:1089-93, 2001; Puglielli L, et al., Nature Cell Biology, 3:905-912, 2001). Cholesterol depletion inhibits the production of Ab in vitro (Simons M, et al., Proc Natl Acad Sci USA, 95:6460-4, 1998), and such cholesterol-lowering drugs as statins reduce the levels of Ab in vitro and in vivo (Fassbender K, et al., Proc Natl Acad Sci USA, 98:5856-61, 2001). Moreover, clinical observations suggest that statins reduce the risk for dementia and AD (lick H, et al., Lancet, 356:1627-31, 2000; Wolozin B, et al., Arch Neurol, 57:1439-43, 2000). Cholesterol 24-hydroxylase is a key enzyme involved in cholesterol removal from brain (Lund E G, et al., Proc Natl Acad Sci USA, 96:7238-43, 1999); it catalyzes the conversion of cholesterol to 24S-hydroxycholesterol (24-OH-Chol), which readily crosses the blood-brain-barrier (Lutjohann D, et al., Proc Natl Acad Sci USA, 93:9799-804, 1996). Hydroxylation is therefore the rate limiting step in cholesterol removal from brain (Bjorkhem I, et al., J Biol Chem, 272:30178-84, 1997; ibid, J Lipid Res, 39:1594-600, 1998). The gene encoding cholesterol 24-hydroxylase, CYP46, is a member of the cytochrome P450 subfamily; it maps to chromosome 14q32.1; GenBank accession number XM 007242. In humans, CYP46 is expressed predominantly in the brain, with mRNA mainly found in the gray matter. In situ hybridizations of mouse brains showed abundant mRNA in neurons of the cerebral cortex, hippocampus, dentate gyrus, and the thalamus.

It is crucial to expand the pool of potential drug targets and diagnostic markers. Therefore, it is an object of the present invention to provide methods of diagnosing or prognosticating Alzheimer's disease. A further objective of the present invention was to provide methods of monitoring the progression of this disease and of evaluating a treatment for Alzheimer's disease. This objective was based on the identification of the CYP46 gene as a novel genetic risk factor that links cholesterol metabolism to Alzheimer's disease. The objective of the present invention has been solved by the methods and kits according to the features of the independent claims. Further preferred embodiments of the present invention are defined in the sub-claims thereto.

The term “and/or” as used in the present specification and in the claims implies that the phrases before and after this term are to be considered either as alternatives or in combination. For instance, the wording “determination of a level and/or an activity” means that either only a level, or only an activity, or both a level and an activity are determined. The term “level” as used herein is meant to comprise a gage of, or a measure of the amount of, or a concentration of a transcription product, for instance an mRNA, or a translation product, for instance a protein or polypeptide. The term “activity” as used herein shall be understood as a measure for the ability of a transcription product or a translation product to produce a biological effect or a measure for a level of biologically active molecules. The term “activity” also refers to enzymatic activity. The terms “level” and/or “activity” as used herein further refer to gene expression levels or gene activity. Gene expression can be defined as the utilization of the information contained in a gene by transcription and translation leading to the production of a gene product. A gene product comprises either RNA or protein and is the result of expression of a gene. The amount of a gene product can be used to measure how active a gene is. The term “gene” as used in the present specification and in the claims comprises both coding regions (exons) as well as non-coding regions (e.g. non-coding regulatory elements such as promoters or enhancers, introns, leader and trailer sequences). Regulatory elements as used in the present disclosure may include inducible and non-inducible promotors, enhancers, operators and other elements that drive and regulate gene expression.

The term “fragment” as used herein is meant to comprise e.g. an alternatively spliced, or truncated, or otherwise cleaved transcription product or translation product. The term “derivative” as used herein refers to a mutant, or an RNA-edited, or a chemically modified, or otherwise altered transcription product, and to a mutant, or chemically modified, or otherwise altered translation product. For instance, a “derivative” may be generated by processes such as altered phosphorylation, or glycosylation, or lipidation, or by altered signal peptide cleavage or other types of maturation cleavage. These processes may occur post-translationally. The term “modulator” as used in the present invention and in the claims refers to a molecule capable of changing or altering the level and/or the activity of a gene, or a transcription product of a gene, or a translation product of a gene. Preferably, a “modulator” is capable of changing or altering the biological activity of a translation product of a gene. Said modulation may be an increase or a decrease in enzyme activity, a change in binding characteristics, or any other change or alteration in the biological, functional, or immunological properties of said translation product of a gene. The singular forms “a”, “an”, and “the” as used herein and in the claims can include plural reference unless the context dictates otherwise. For example “a cell” may also mean a plurality of cells.

In one aspect, the invention features a method for diagnosing or prognosticating Alzheimer's disease in a subject, or determining the propensity or predisposition of a subject to develop Alzheimer's disease. The method comprises detecting in a sample obtained from said subject the presence or absence of a variation in CYP46 gene, wherein the presence of a variation in the CYP46 gene in said subject indicates a diagnosis or prognosis of Alzheimer's disease, or an increased propensity or predisposition of developing Alzheimer's disease as compared to a subject who does not carry a variation in said gene. The CYP46 gene codes for the enzyme cholesterol 24-hydroxylase. The GenBank accession number of CYP46 is XM 007242. The terms “propensity” or “predisposition” as employed herein are used interchangeably with reference to developing Alzheimer's disease and are tantamount to the terms “susceptibility” or “risk”. A variation in a CYP46 gene can be understood as any alteration in the naturally occuring nucleic acid sequence of the CYP46 gene, i.e. any alteration from the wildtype.

In a preferred embodiment, the variation in the CYP46 gene is a single nucleotide polymorphism at a position 151 bp 5′ of exon 3 (single nucleotide polymorphism identification number: rs754203. In a further preferred embodiment, the variation is a C to T transition.

In a preferred embodiment of the invention, said variation is present in both copies of the CYP46 gene. This means that the subject is homozygous for the said variation. The genotype of said subject is then herein designated as CYP46*TT.

The method, according to the present invention, may be particularly useful for the identification of individuals that are at risk of developing Alzheimer's disease. Consequently, the method, according to the present invention, may serve as a means for targeting identified individuals for early preventive measures or therapeutic intervention prior to disease onset, before irreversible damage in the course of the disease has been inflicted.

Determining the presence or absence of a polymorphism or variation in a CYP46 gene may comprise determining a partial nucleotide sequence of the DNA from said subject, said partial nucleotide sequence indicating the presence or absence of said polymorphism or variation. It may further be preferred to perform a polymerase chain reaction with the DNA from said subject to determine the presence or absence of said polymorphism or variation. Such techniques are known to those skilled in the art (see Lewin B, Genes V, Oxford University Press, 1994).

In a preferred embodiment of the invention, the method further comprises detecting in a sample from said subject the presence of an apolipoprotein E4 allele, wherein the presence of both the variation in the CYP46 gene in both copies of the gene (in other words, wherein said subject is homozygous for said variation; the CYP46*TT genotype) and the presence of an apolipoprotein E4 allele in said subject indicates a diagnosis or prognosis of Alzheimer's disease, or a further increased propensity or predisposition to develop Alzheimer's disease as compared to a subject who carries either only said variation in the CYP46 gene or only an apolipoprotein E4 allele, or neither said variation in the CYP46 gene and an apolipoprotein E4 allele. The method of this embodiment reflects the surprising finding of an unexpected synergistic interaction between the genes for CYP46*T and apolipoprotein E4.

In a preferred embodiment of the invention, the sample taken for genetic analysis comprises DNA obtained from body fluids, tissues, or any suitable cells of the body readily available. Preferably, the sample is a blood sample. However, the sample may also consist of body fluids such assaliva, urine, serum plasma, nasal mucosa, or cerebrospinal fluid. In a further aspect, the invention features a method for diagnosing or prognosticating Alzheimer's disease in a subject, or determining the propensity or predisposition of a subject to develop Alzheimer's disease, comprising: determining a level, or an activity, or both said level and said activity, of at least one substance which is selected from the group consisting of a transcription product of the CYP46 gene or a translation product of the CYP46 gene in a sample from said subject; and comparing said level, or said activity, or both said level and said activity, of at least one of said substances to a reference value representing a known disease or health status, thereby diagnosing or prognosticating Alzheimer's disease in said subject, or determining the propensity or predisposition of said subject to develop Alzheimer's disease.

In another aspect, the present invention provides a method of monitoring the progression of Alzheimer's disease in a subject, comprising: determining a level, or an activity, or both said level and said activity, of at least one substance which is selected from the group consisting of a transcription product of the CYP46 gene or a translation product of the CYP46 gene in a sample from said subject; and comparing said level, or said activity, or both said level and said activity, of at least one of said substances to a reference value representing a known disease or health status, thereby monitoring the progression of Alzheimer's disease in said subject.

In a further aspect, the present invention provides a method of evaluating a treatment for Alzheimer's disease, comprising: determining a level, or an activity, or both said level and said activity, of at least one substance which is selected from the group consisting of a transcription product of the CYP46 gene or a translation product of the CYP46 gene in a sample obtained from a subject being treated for Alzheimer's disease; and comparing said level, or said activity, or both said level and said activity, of at least one of said substances to a reference value representing a known disease or health status, thereby evaluating said treatment for Alzheimer's disease.

In a preferred embodiment of the invention, the sample to be analyzed for determining a level, or an activity, or both said level and said activity, of at least one substance which is selected from the group consisting of a transcription product of the CYP46 gene or a translation product of the CYP46 gene, is taken from a body fluid, preferably cerebrospinal fluid, saliva, urine, nasal mucosa, or blood, or serum plasma, or a tissue, or cells like skin fibroblasts. Most preferably, the sample is taken from cerebrospinal fluid.

In a preferred embodiment of the invention, the reference value of a level, or an activity, or both said level and said activity, of a transcription product of the CYP46 gene or a translation product of the CYP46 gene, is that in a sample from a subject not suffering from Alzheimer's disease.

The determination of a level of transcription products of a CYP46 gene can be performed in a sample from a subject using Northern blots with probes specific for said gene. Another preferred method of measuring said level is by quantitative PCR with primer combinations which amplify said gene-specific sequences from cDNA obtained by reverse transcription of RNA extracted from a sample of a subject. Another preferred method for the analysis of transcription products is chip based microarray-technology. These techniques are known to those of ordinary skill in the art (see Sambrook and Russell, Molecular Cloning: A Laboratory Manual, Cold Spring Harbor Laboratory Press, Cold Spring Harbor, N.Y., 2000). Furthermore, the level and/or activity of a translation product of the CYP46 gene (e.g. a cholesterol 24-hydroxylase polypeptide) can be detected using a Western blot analysis, an immunoassay, an ezyme activity assay, and/or binding assay. These assays can measure the amount of binding between said translation product and an anti-polypeptide antibody by the use of enzymatic, chromodynamic, radioactive, or luminescent labels which are attached to either the anti-polypeptide antibody or a secondary antibody which binds the anti-polypeptide antibody. In addition, other high affinity ligands may be used. Immunoassays which can be used include e.g. ELISAs, Western blots and other techniques known to those of ordinary skill in the art (see Harlow and Lane, Antibodies: A Laboratory Manual, Cold Spring Harbor Laboratory Press, Cold Spring Harbor, N.Y., 1999). is Translation products may also be assayed by protein-chip based technologies. Since the CYP46 gene encodes the enzyme cholesterol 24-hydroxylase, its enzymatic activity may be measured by in vitro, cell-based, or in vivo assays. Conveniently, cholesterol 24-hydroxylase enzymatic activity can, for instance, be determined using a hydroxylase activity assay.

In a preferred embodiment, the provided methods of diagnosing or prognosticating Alzheimer's disease in a subject, or determining the propensity or predisposition of a subject to develop Alzheimer's disease, or monitoring a treatment, or evaluating a treatment of Alzheimer's disease further comprise comparing a level, or an activity, or both said level and said activity, of a transcription product of the CYP46 gene or a translation product of the CYP46 gene, in a series of samples taken from said subject over a period of time. In another preferred embodiment, said subject receives a treatment prior to one or more sample gatherings. It is a further preferred embodiment to determine said level, or said activity, or both said level and said activity, in said samples before and after said treatment of said subject.

In another aspect, the invention features a kit for diagnosing or prognosticating Alzheimer's disease in a subject, or determining the propensity or predisposition of a subject to develop Alzheimer's disease, said kit comprising:
(a) at least one reagent which is selected from the group consisting of (i) reagents that selectively detect a transcription product of the CYP46 gene, (ii) reagents that selectively detect a translation product of the CYP46 gene, (iii) reagents that selectively detect the presence or absence of a variation in the CYP46 gene; and
(b) instruction for diagnosing, or prognosticating Alzheimer's disease, or determining the propensity or predisposition of a subject to develop Alzheimer's disease by
   detecting a level, or an activity, or both said level and said activity, of said transcription product and/or said translation product of the CYP46 gene, in a sample from said subject; and/or detecting the presence or absence of a variation in the CYP46 gene in a sample from said subject; and
   diagnosing or prognosticating Alzheimer's disease, or determining the propensity or predisposition of said subject to develop Alzheimer's disease,
      wherein a varied level, or activity, or both said level and said activity, of said transcription product and/or said translation product compared to a reference value representing a known health status; or a level, or activity, or both said level and said activity, of said transcription product and/or said translation product similar or equal to a reference value representing a known disease status; or the presence of a variation in the CYP46 gene indicates a diagnosis or prognosis of Alzheimer's disease, or an increased propensity or predisposition of developing Alzheimer's disease.

It is preferred that the reagents of the kit selectively detect the single nucleotide polymorphism at the position 151 bp 5′ of exon 3 (single nucleotide polymorphism identification number: rs754203) in the CYP46 gene. It is further preferred that the variation is a C to T transition. It is further preferred, that for the purpose of diagnosing or prognosticating Alzheimer's disease in said subject, or determining the propensity or predispositon of said subject to develop Alzheimer's disease, said subject is homozygous in respect to said variation.

In another preferred embodiment, the kit further comprises reagents that selectively detect the presence or absence of an apolipoprotein E4 allele. The presence of an apolipoprotein E4 allele indicates a diagnosis or prognosis of Alzheimer's disease, or a further increased propensity or is predisposition of developing Alzheimer's disease. This embodiment reflects the unexpected synergistic interaction between the alleles for CYP46*T and an apolipoprotein E4 allele.

The kit, according to the present invention, may be particularly useful for the identification of individuals that are at risk of developing Alzheimer's disease. Consequently, the kit, according to the invention, may serve as a means for targeting identified individuals for early preventive measures or therapeutic intervention prior to disease onset, before irreversible damage in the course of the disease has been inflicted.

Furthermore, in preferred embodiments, the kit featured in the invention is useful for monitoring a progression of Alzheimer's disease in a subject. It is further useful in monitoring success or failure of therapeutic treatment of said subject.

In another aspect, the invention features a method of treating or preventing Alzheimer's disease or related neurodegenerative diseases, in a subject comprising the administration to said subject in a therapeutically or prophylactically effective amount of an agent or agents which directly or indirectly affect a level, or an activity, or both said level and said activity, of (i) the CYP46 gene, and/or (ii) a transcription product of the CYP46 gene, and/or (iii) a translation product of the CYP46 gene, and/or (iv) a fragment or derivative of (i) to (iii).

In the context of the present invention, Alzheimer's disease related dementias and neurodegenerative diseases are, for instance, Parkinson's. disease, Huntington's disease, amyotrophic lateral sclerosis, Pick's disease, fronto-temporal dementia, progressive nuclear palsy, cerebro-vascular dementia, or corticobasal degeneration. Further conditions involving neurodegenerative processes are, for instance, ischemic stroke and age-related macular degeneration.

In preferred embodiments, the method comprises the application of per se known methods of gene therapy and/or antisense nucleic acid technology to administer said agent or agents. In general, gene therapy comprises several approaches: molecular replacement of a mutated gene, addition of a new gene resulting in the synthesis of a therapeutic protein, and modulation of endogenous cellular gene expression by recombinant expression methods or by drugs. Gene-transfer techniques are described in detail (see e.g. Behr, Acc Chem Res 1993, 26:274-278 and Mulligan, Science, 1993, 260: 926-931) and include direct gene-transfer techniques such as mechanical microinjection of DNA into a cell as well as indirect techniques employing biological vectors (like recombinant viruses, especially retroviruses) or model liposomes, or techniques based on transfection with DNA coprecipitation with polycations, cell membrane pertubation by chemical (solvents, detergents, polymers, enzymes) or physical means (mechanic, osmotic, thermic, electric shocks). The postnatal gene transfer into the central nervous system has been described in detail (see e.g. Wolff, Curr Opin Neurobiol 1993, 3:743-748).

In particular, the invention features a method of treating or preventing a neurodegenerative disease by means of antisense nucleic acid therapy, i.e. the down-regulation of an inappropriately expressed or defective gene by the introduction of antisense nucleic acids or derivatives thereof into certain critical cells (see e.g. Gillespie, DN&P 1992, 5:389-395; Agrawal and Akhtar, Trends Biotechnol 1995, 13:197-199; Crooke, Biotechnology 1992, 10:882-6). Apart from hybridization strategies, the application of ribozymes, i.e. RNA molecules that act as enzymes, destroying RNA that carries the message of disease has also been described (see e.g. Barinaga, Science 1993, 262:1512-1514). In preferred embodiments, the subject to be treated is a human, and therapeutic antisense nucleic acids or derivatives thereof are directed against the human CYP46 gene. It is preferred that cells of the central nervous system, preferably the brain, of a subject are treated in such a way. Cell penetration can be performed by known strategies such as coupling of antisense nucleic acids and derivatives thereof to carrier particles, or the above described techniques. Strategies for administering targeted therapeutic oligodeoxynucleotides are known to those of skill in the art (see e.g. Wickstrom, Trends Biotechnol, 1992, 10: 281-287). In some cases, delivery can be performed by mere topical application. Further approaches are directed to intracellular expression of antisense RNA. In this strategy, cells are transformed ex vivo with a recombinant gene that directs the synthesis of an RNA that is complementary to a region of target nucleic acid. Therapeutical use of intracellularly expressed antisense RNA is procedurally similar to gene therapy.

In further preferred embodiments, the method comprises grafting donor cells into the central nervous system, preferably the brain, of said subject, or donor cells preferably treated so as to minimize or reduce graft rejection, wherein said donor cells are genetically modified by insertion of at least one transgene encoding said agent or agents. Said transgene might be carried by a viral vector, in particular a retroviral vector. The transgene can be inserted into the donor cells by a nonviral physical transfection of DNA encoding a transgene, in particular by microinjection. Insertion of the transgene can also be performed by electroporation, chemically mediated transfection, in particular calcium phosphate transfection or liposomal mediated transfection.

In preferred embodiments, said agent is a therapeutic protein which can be administered to said subject, preferably a human, by a process comprising introducing subject cells into said subject, said subject cells having been treated in vitro to insert a DNA segment encoding said therapeutic protein, said subject cells expressing in vivo in said subject a therapeutically effective amount of said therapeutic protein. Said DNA segment can be inserted into said cells in vitro by a viral vector, in particular a retroviral vector. Said agent, particularly a therapeutic protein, can further be administered to said subject by a process comprising the injection or the systemic administration of a fusion protein, said fusion protein consisting of a fusion of a protein transduction domain with said agent.

Methods of treatment, according to the present invention, comprise the application of therapeutic cloning, transplantation, and stem cell therapy using embryonic stem cells or embryonic germ cells and neuronal adult stem cells, combined with any of the previously described cell- and gene therapeutic methods. Stem cells may be totipotent or pluripotent. They may also be organ-specific. Strategies for repairing diseased and/or damaged brain cells or tissue comprise (i) taking donor cells from an adult tissue. Nuclei of those cells are transplanted into unfertilized egg cells from which the genetic material has been removed. Embryonic stem cells are isolated from the blastocyst stage of the cells which underwent somatic cell nuclear transfer. Use of differentiation factors then leads to a directed development of the stem cells to specialized cell types, preferably neuronal cells (Lanza et al., Nature Medicine 1999, 9: 975-977), or (ii) purifying adult stem cells, isolated from the central nervous system, or from bone marrow (mesenchymal stem cells), for in vitro expansion and subsequent grafting and transplantation, or (iii) directly inducing endogenous neural stem cells to proliferate, migrate, and differentiate into functional neurons (Peterson D A, Curr. Opin. Pharmacol. 2002, 2:. 34-42). Adult neural stem cells are of great potential for repairing damaged or diseased brain tissues, as the germinal centers of the adult brain are free of neuronal damage or dysfunction (Colman A, Drug Discovery World 2001, 7: 66-71).

In preferred embodiments, the subject for treatment or prevention, according to the present invention, can be a human, an experimental animal, e.g. a mammal, a mouse, a rat, a fish, a fly, or a worm; a domestic animal, or a non-human primate. The experimental animal can be an animal model for a neurodegenerative disorder, e.g. a transgenic mouse with an Alzheimer's-type neuropathology.

In a further aspect, the invention features a modulator of an activity, or a level, or both said activity and said level of at least one substance which is selected from the group consisting of (i) the CYP46 gene and/or (ii) a transcription product of the CYP46 gene, and/or (iii) a translation product of the CYP46 gene, and/or (iv) a fragment or derivative of (i) to (iii).

In an additional aspect, the invention features a pharmaceutical composition comprising said modulator and preferably a pharmaceutical carrier. Said carrier refers to a diluent, adjuvant, excipient, or vehicle with which the modulator is administered.

In a further aspect, the invention features a modulator of an activity, or a level, or both said activity and said level of at least one substance which is selected from the group consisting of (i) the CYP46 gene, and/or (ii) a transcription product of the CYP46 gene and/or (iii) a translation product of the CYP46 gene, and/or (iv) a fragment or derivative of (i) to (iii) for use in a pharmaceutical composition.

In another aspect, the invention provides for the use of a modulator of an activity, or a level, or both said activity and said level of at least one substance which is selected from the group consisting of (i) the CYP46 gene, and/or (ii) a transcription product of the CYP46 gene, and/or (iii) a translation product of the CYP46 gene, and/or (iv) a fragment or derivative of (i) to (iii) for a preparation of a medicament for treating or preventing a neurodegenerative disease, in particular Alzheimer's disease.

In one aspect, the present invention also provides a kit comprising one or more containers filled with a therapeutically or prophylactically effective amount of said pharmaceutical composition.

In a further aspect, the invention features a recombinant, non-human animal comprising a non-native gene sequence coding for a translation product of the CYP46 gene, or a fragment thereof, or a derivative thereof. The generation of said recombinant, non-human animal comprises (i) providing a gene targeting construct containing said gene sequence and a selectable marker sequence, and (ii) introducing said targeting construct into a stem cell of a non-human animal, and (iii) introducing said non-human animal stem cell into a non-human embryo, and (iv) transplanting said embryo into a pseudopregnant non-human animal, and (v) allowing said embryo to develop to term, and (vi) identifying a genetically altered non-human animal whose genome comprises a modification of said gene sequence in both alleles, and (vii) breeding the genetically altered non-human animal of step (vi) to obtain a genetically altered non-human animal whose genome comprises a modification of said endogenous gene, wherein said gene is mis-expressed, or under-expressed, or over-expressed, and wherein said disruption or alteration results in said non-human animal exhibiting a predisposition to developing symptoms of neuropathology similar to a neurodegenerative disease, in particular Alzheimer's disease. Strategies and techniques for the generation and construction of such an animal are known to those of ordinary skill in the art (see e.g. Capecchi, Science, 1989, 244:1288-1292 and Hogan et al., 1994, Manipulating the Mouse Embryo: A Laboratory Manual, Cold Spring Harbor Laboratory Press, Cold Spring Harbor, N.Y.). It is preferred to make use of such a recombinant non-human animal as an animal model for investigating neurodegenerative diseases, in particular Alzheimer's disease.

In another aspect, the invention features an assay for screening for a modulator of neurodegenerative diseases, in particular Alzheimer's disease, or related diseases and disorders of one or more substances selected from the group consisting of (i) the CYP46 gene, and/or (ii) a transcription product of the CYP46 gene, and/or (iii) a translation product of the CYP46 gene, and/or (iv) a fragment or derivative of (i) to (iii). This screening method comprises (a) contacting a cell with a test compound, and (b) measuring the level, or the activity, or both the level and the activity of one or more substances recited in (i) to (iv), and (c) measuring the level, or the activity, or both the level and the activity of said substances in a control cell not contacted with said test compound, and (d) comparing the levels of the substance in the cells of step (b) and (c), wherein an alteration in the level and/or activity of said substances in the contacted cells indicates that the test compound is a modulator of said diseases and disorders.

In one further aspect, the invention features a screening assay for a modulator of neurodegenerative diseases, in particular Alzheimer's disease, or related diseases and disorders of one or more substances selected from the group consisting of (i) the CYP46 gene, and/or (ii) a transcription product of the CYP46 gene, and/or (iii) a translation product of the CYP46 gene, and/or (iv) a fragment or derivative of (i) to (iii), comprising (a) administering a test compound to a test animal which is predisposed to developing or has already developed a neurodegenerative disease or related diseases or disorders, and (b) measuring the level and/or activity of one or more substances recited in (i) to (iv), and (c) measuring the level and/or activity of said substances in a matched control animal which is equally predisposed to developing or has already developed said diseases and to which animal no such test compound has been administered, and (d) comparing the level and/or activity of the substance in the animals of step (b) and (c), wherein an alteration in the level and/or activity of substances in the test animal indicates that the test compound is a modulator of said diseases and disorders.

In a preferred embodiment, said test animal and/or said control animal is a recombinant, non-human animal which expresses the CYP46 gene, or a fragment thereof, or a derivative thereof, under the control of a transcriptional regulatory element which is not the native CYP46 gene transcriptional control regulatory element.

In another embodiment, the present invention provides a method for producing a medicament comprising the steps of (i) identifying a modulator of neurodegenerative diseases by a method of the herein aforementioned screening assays and (ii) admixing the modulator with a pharmaceutical carrier. However, said modulator may also be identifiable by other types of screening assays.

In another aspect, the present invention provides for a method of testing a compound, preferably an assay for screening a plurality of compounds, for inhibition of binding between a ligand and a CYP46 gene product, or a fragment or derivative thereof. Said method comprises the steps of (i) adding a liquid suspension of said CYP46 gene product, or a fragment or derivative thereof, to a plurality of containers, and (ii) adding a compound, preferably a plurality of compounds, to be screened for said inhibition to said plurality of containers, and (iii) adding detectable ligand, preferably fluorescently detectable ligand, to said containers, and (iv) incubating the liquid suspension of said CYP46 gene product, or said fragment or derivative thereof, and said compounds, and said detectable ligand, and (v) measuring the amounts of detectable ligand or fluorescence associated with said CYP46 gene product, or with said fragment or derivative thereof, and (vi) determining the degree of inhibition by one or more of said compounds of binding of said ligand to said CYP46 gene product, or said fragment or derivative thereof. Instead of utilizing a fluorescently detectable label, it might in some aspects be preferred to use any other detectable label known to the person skilled in the art, e.g. radioactive label, and detect it accordingly. Said method may be useful for the identification of novel compounds as well as for evaluating compounds which have been improved or otherwise optimized in their ability to inhibit the binding of a ligand to a CYP46 gene product, or a fragment or derivative thereof. In one further embodiment, the present invention provides a method for producing a medicament comprising the steps of (i) identifying a compound as an inhibitor of binding between a ligand and a CYP46 gene product by the herein aforementioned inhibitory binding assay and (ii) admixing the compound with a pharmaceutical carrier. However, said compound may also be identifiable by other types of screening assays.

In one further aspect, the invention features a method of testing a compound, preferably an assay for screening a plurality of compounds, to determine the degree of binding of said compound or compounds to a CYP46 gene product, or to a fragment or derivative thereof. Said method comprises the steps of (i) adding a liquid suspension of said CYP46 gene product, or a fragment or derivative thereof, to a plurality of containers, and (ii) adding a detectable compound, preferably a plurality of detectable compounds, in particular fluorescently detectable compounds, to be screened for said binding to said plurality of containers, and (iii) incubating the liquid suspension of said CYP46 gene product, or said fragment or derivative thereof, and said detectable compound, preferably said plurality of detectable compounds, and (iv) measuring the amounts of detectable compound or fluorescence associated with said CYP46 gene product, or with said fragment or derivative thereof, and (v) determining the degree of binding by one or more of said compounds to said CYP46 gene product, or said fragment or derivative thereof. In this type of assay it might be preferred to use a fluorescent label. However, any other type of detectable label might also be employed. Said method may be useful for the identification of novel compounds as well as for evaluating compounds which have been improved or otherwise optimized in their ability to bind to a CYP46 gene product. In one further embodiment, the present invention provides a method for producing a medicament comprising the steps of (i) identifying a compound as a binder to a CYP46 gene product by the herein aforementioned binding assays and (ii) admixing the compound with a pharmaceutical carrier. However, said compound may also be identifiable by other types of screening assays.

In another embodiment, the present invention provides for a medicament obtainable by any of the methods according to the herein claimed screening assays. In one further embodiment, the instant invention provides for a medicament obtained by any of the methods according to the herein claimed screening assays.

The present invention features an antibody which is specifically immunoreactive with an immunogen, wherein said immunogen is a translation product of the CYP46 gene or a fragment thereof. The immunogen may comprise immunogenic or antigenic epitopes of portions of a translation product of said genes, wherein said immunogenic or antigenic portion of a translation product is a polypeptide, and wherein said polypeptide elicits an antibody response in an animal, and wherein said polypeptide is immunospecifically bound by said antibody. Methods for generating antibodies are well known in the art (see Harlow et al., Antibodies, A Laboratory Manual, Cold Spring Harbor Laboratory Press, Cold Spring Harbor, N.Y.). The term “antibody” encompasses all forms of antibodies known in the art, such as polyclonal, monoclonal, chimeric, recombinatorial, single chain antibodies as well as fragments thereof. Antibodies of the present invention are useful, for instance, in a variety of diagnostic and therapeutic methods involving detecting translation products of the CYP46 gene.

In a preferred embodiment of the present invention, said antibodies can be used for detecting the pathological state of a cell in a sample from a subject, comprising immunocytochemical staining of said cell with said antibody, wherein an altered degree of staining, or an altered staining pattern in said cell compared to a cell representing a known health status indicates a pathological state of said cell. Preferably, the pathological state relates to a neurodegenerative disease, in particular to Alzheimer's disease. Immuno-cytochemical staining of a cell can be carried out by a number of different experimental methods well known in the art. It might be preferred, however, to apply an automated method for the detection of antibody binding, wherein the determination of the degree of staining of a cell, or the determination of the cellular or subcellular staining pattern of a cell, or the topological distribution of an antigen on the cell surface or among organelles and other subcellular structures within the cell, are carried out according to the methods described in the U.S. Pat. No. 6,150,173.

Other features and advantages of the invention will be apparent from the following detailed description of the figures and examples, which are illustrative only, and not intended to limit the remainder of the disclosure in any way.

Table 1: shows CYP46 genotype and allele distribution in control subjects and Alzheimer's disease patients.

Table 2: shows the unconditional logistic regression analysis (forward is and backward) with the diagnosis of Alzheimer's disease as a dependent variable.

Table 3: shows the interaction between APOE and CYP46 genotypes and risk for Alzheimer's disease (combined sample).

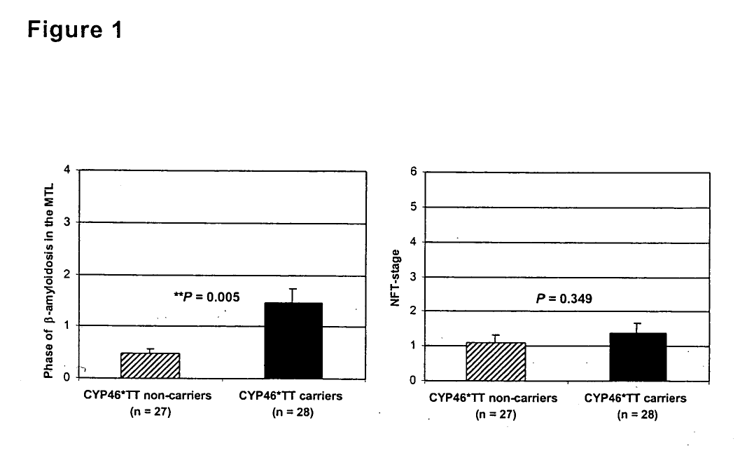

FIG. 1: depicts the mean phase of b-amyloidosis in the medial temporal lobe (left panel) and NFT-staging (right panel) in non-demented elderly carriers (solid bars) and non-carriers (hatched bars) of the CYP46*TT genotype. Error bars indicate standard error of the mean (SEM).

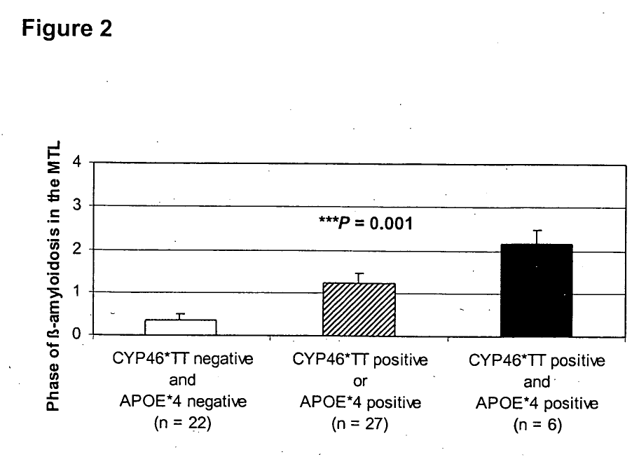

FIG. 2: illustrates the differences in the phase of b-amyloidosis in the medial temporal lobe due to interaction between APOE*4 and CYP46*TT (mean±SEM).

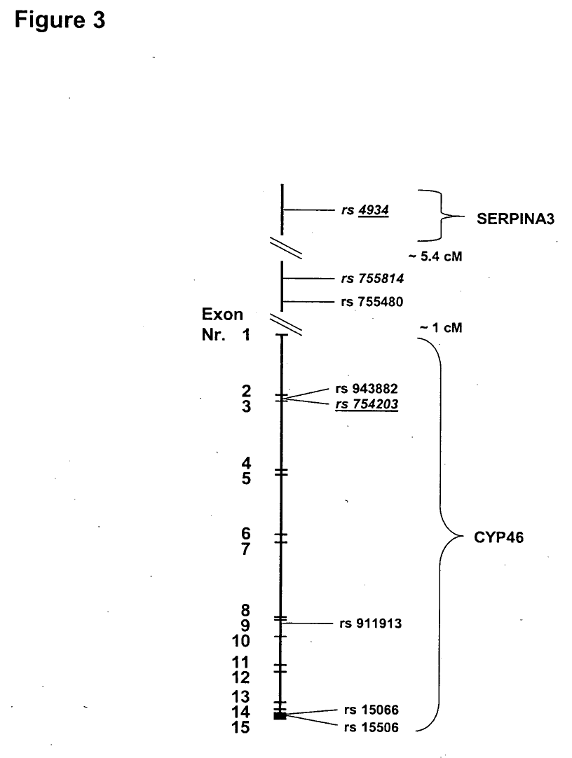

FIG. 3: depicts a schematic representation of the studied genomic region and SNPs on chromosome 14q. Only SNPs in italics proved to be polymorphic in a sample of 50 individuals. The underlined SNPs -(rs4937 and rs754203) were used in the present study. SERPINA3 encodes a1-antichymotrypsin, CYP46 encodes cholesterol 24-hydroxylase. SNP information was derived from the database of single nucleotide polymorphisms (dbSNP) established by the National Center for Biotechnology Information (http://www.ncbi.nlm.nih.gov/SNP/index.html).

### EXAMPLE 1

To determine whether polymorphisms of CYP46 may be associated with an increased risk for AD, we performed a population-based case-control association study to test for allelic and genotype differences of a single nucleotide polymorphism (SNP) of CYP46 between patients with late-onset AD and non-demented control subjects. Population-based case-control association studies may be prone to false-positive findings related to genetic heterogeneity, selection and ascertainment bias, and multiple testing. It has been proposed that population-based genetic association studies should be performed and replicated in independent populations, have small P values and high odds ratios, and report biologically meaningful associations. Therefore, we sampled DNA in two separate and independent populations: a hypothesis testing population from Northern Greece (Thessaloniki) and Italy (Brescia) and a hypothesis confirming Swiss-German population (Zurich). To characterize possible effects of CYP46 on brain amyloid formation, we tested the association with Ab deposition in control subjects that had no other neuropathological abnormalities, and we quantified the CSF levels of Ab42 in AD patients.

The genotype distribution of CYP46 was that expected under Hardy-Weinberg equilibrium both in patients with AD and in control subjects in both samples (p≧0.45 for each comparison). Genotype analysis in the hypothesis testing sample revealed a significantly higher frequency of the CYP46*TT genotype in patients with AD as compared to control subjects (60.7% vs 46.1% respectively, p=0.049) (Table 1). A significant difference was also observed in the hypothesis confirming sample, with frequencies for the CYP46*TT genotype of 57.9% in patients with AD and 42.0% in control subjects (p=0.031). The distribution of the CYP46 genotype between control subjects of the hypothesis testing and the hypothesis confirming sample and between patients with AD of these samples was similar (p=0.658 and p=0.698, respectively), therefore, both samples were combined to increase statistical power and to allow for the analysis of additional variables and genetic interactions. Forward and backward unconditional logistic regression analysis was performed for the simultaneous assessment of the influence of age, gender, APOE, and CYP46 genotype on the risk for developing AD (Table 2). Age was included as a continuous variable (1-year intervals). Gender, APOE, and CYP46 genotypes were binomial categorial variables. The analysis of the combined sample revealed a significant influence of the APOE and CYP46 genotypes on the risk for AD (p<0.00001 and p=0.0004, respectively), but no influence of either age or gender (p=0.97 and p=0.08, respectively). The adjusted odds ratio for the development of AD in APOE*4-carriers was 4.06, the corresponding value for homozygous carriers of the CYP46*T allele was 2.19. Separate analysis in the hypothesis testing and hypothesis confirming sample yielded similar results (Table 2).

We determined a significant synergistic interaction between APOE and CYP46 (Table 3): When compared to subjects carrying neither the APOE*4 allele nor the CYP46*TT genotype, the odds ratio for subjects carrying the CYP46*TT genotype and the APOE*4 allele was 9.07 (95% CI: 4.37-19.07, p=2.2*10⁻¹⁰). The corresponding odds ratio for APOE*4 carriers without the CYP46*TT genotype was 3.70 (95% CI: 1.92-7.16, p=0.00002) and 2.03 (95% CI: 1.13-3.67, p=0.012) for CYP46*TT genotype carriers without the APOE*4 allele.

We determined the amyloid load in the medial temporal lobe (MTL) of elderly subjects devoid of significant neuropathological abnormalities other than amyloid deposition and related it to the presence, or the absence, of the CYP46*TT genotype (FIG. 1). Amyloid load was defined as the observed evolutionary phase of b-amyloidosis in the MTL (Thal D R, et al., J Neuropathol Exp Neurol, 59:733-48, 2000). The mean phase of b-amyloidosis in the MTL of CYP46*TT genotype carriers (n=28) was 1.46±1.35, the corresponding mean phase in non-carriers of the CYP46*TT genotype (n=27) was significantly lower (0.48±0.75, p=0.005, two-tailed U-test). Again, a significant interaction between APOE and CYP46 was observed, in that the mean phase of b-amyloidosis in the MTL was lowest in APOE*4 and CYP46*TT negatives, intermediate in subjects positive for either APOE*4 or CYP46*TT, and highest for carriers or both genotypes (FIG. 2). Braak staging for neurofibrillary tangles (NFT) revealed no differences between carriers and non-carriers of the CYP46*TT genotype (p=0.349, two-tailed U-test).

We further investigated whether the CYP46*TT genotype is associated with altered cerebrospinal fluid (CSF) levels of Ab42 in AD patients (n=33) and observed a 35% increase in carriers of the CYP46*TT genotype (n=16) as compared to non-carriers (n=17) (0.31±0.11 ng/ml vs 0.23±0.12 ng/ml, respectively,, p=0.014, two-tailed U-test).

We finally investigated whether the observed association between the CYP46*TT genotype and AD is attributable to linkage disequilibrium (LD) with a distinct locus within CYP46 or in the vicinity of this gene. Sequencing of all 15 exons of CYP46 in 10 elderly individuals homozygote for the CYP46*T allele and 10 CYP46*C allele homozygotes revealed no exonic SNPs. SERPINA3, which encodes the proteinase inhibitor a1-antichymotrypsin maps—like CYP46—to chromosome 14q32.1 and has been previously associated with the development of AD (Kamboh M I, et al., Nat Genet, 10:486-8, 1995). Consequently, we examined whether the SERPINA3*A allele of a common SNP in the signal peptide of a1-antichymotrypsin confers significant risk for AD as originally reported, and whether the SNPs of SERPINA3 and CYP46 are in LD in our sample. SERPINA3 genotyping was performed in 153 AD patients and 181 control subjects of the combined sample. The frequency of the SERPINA3*A allele was 55.0% in control subjects and 55.2% in AD patients (p=0.947). Logistic regression analysis with age, sex, APOE, and SERPINA3 alleles and genotypes as independent variables did not show any significant influence of SERPINA3 on AD risk. In addition, no significant gene-gene interaction between SERPINA3 and CYP46 was observed (p=0.267). No significant LD between SERPINA3 and CYP46 polymorphisms was observed in each sample of patients and control subjects.

Subjects:

Genetic Association Studies:

Genetic studies were conducted on 2 independent Caucasian populations: a hypothesis testing sample (183 participants) and a hypothesis confirming sample (248 participants). The diagnosis of AD was performed according to the NINCDS-ADRDA criteria based on medical interview, physical examination, neuropsychological testing, brain MRI or CT, as well as blood tests. The mean Mini-Mental State Examination (MMSE) score of the overall patient population (n=183) was 19.9, the mean age was 73.9 years, the mean age-at-onset of AD was 70.8 years. There were 110 (60.1%) female participants in the patient group. The control group (n=248) comprised cognitively healthy elderly individuals who were either the spouses of AD patients or subjects recruited from the outpatient clinics of the participating institutions. The mean age was 73.9 years and the mean MMSE score was 29.1. There were 123 (49.6%) female participants in this group.

Neuropathological Studies:

Neuropathological examinations were performed in the brains of 55 elderly individuals (mean age of death: 72.2 years, range 60-91 years, 23 females) devoid of significant neuropathological abnormalities and without signs of dementia as measured by the Clinical Dementia Rating (CDR) scale (Hughes C P, et al., Br J Psychiatry, 140:566-72, 1982). The evolutionary phases (0-4) of b-amyloidosis in the medial temporal lobe of these subjects were determined as described before (Thal D R, et al., J Neuropathol Exp Neurol, 59:733-48, 2000). Neurofibrillary tangle (NFT) staging (0-6) was performed according to Braak and Braak (Acta Neuropathol, 82:239-59, 1991). For the determination of the APOE and CYP46 genotypes, DNA was extracted from cerebellar fresh frozen tissue samples following standard protocols known to the skilled person.

CSF Studies:

Ab42 concentration was determined in the CSF of 33 AD patients. All participants were recruited in Zurich. The mean age was 71.5 years, the mean MMSE score was 21.9. CSF was obtained by lumbar puncture according to conventional techniques. CSF samples were frozen on dry ice immediately upon withdrawal at the bedside in 0.5 ml aliquots and stored at −85° C. until biochemical analyses.

Methods:

SNP Selection and Genotyping:

Information on polymorphic sites of CYP46 and SERPINA3 was derived from the database of single nucleotide polymorphisms (dbSNP) established by the National Center for Biotechnology Information (http://www.ncbi.nlm.nih.gov/SNP/index.html). Five SNPs within the genomic region of CYP46 and 2 SNPs 1cM 5′ to exon 1 of CYP46 were selected for genotyping (FIG. 3). Of these 7 potential SNPs, only 2 (rs754203 and rs755814) proved to be polymorphic in a sub-sample of 50 participants. rs754203 is located 151 bases 5′ to exon 3 of CYP46 and predicts a T to C base exchange. rs755814 was not used for subsequent genotyping of the entire sample, since the frequency of the minor allele was too low (<1%).

SNPs rs754203 and rs4934 (on SERPINA3) were genotyped by the pyrosequencing™ method (www.pyrosequencing.com) on a PSQ™ 96 System. Forward and backward amplification primers for rs754203 were 5′-AAT GCA TGC TAC CAA AAG AG-3′ and 5′-AAT CAT TTG ATT CCC AGG AC-3′, respectively. The backward primer was biotinylated at the 3′ end. Sequencing primer was 5′-GGC AGA GCC TTG CCC-3′. Forward and backward amplification primers for rs4934 were 5′-CAG AGT TGA GAA TGG AGA-3′ and 5′- TTC TCC TGG GTC AGA TTC -3′, respectively. The backward primer was biotinylated at the 3′ end. Sequencing primer was 5′-GGA GAG AAT GTT ACC TCT C-3′. APOE genotyping was performed according to Hixson and Vernier (J Lipid Res, 31:545-8, 1990).

Analysis Ab42 in the CSF:

CSF Ab42 levels were determined using a sandwich ELISA (INNOTEST b-Amyloid 1-42, Innogenetics). The monoclonal antibody 21F12—specific for the free C-terminal end of Ab42 (peptide sequence Ab33-42)—was used as capturing antibody, while the biotinylated monoclonal antibody 3D6—specific for the N-terminal end of Ab42 (peptide sequence Ab1-5)—was used as detector. Absorbance was read at 450 nm on a microplate reader (Victor2 Multilabel, EG&GO Wallac). The linear range of the assay was 50 pg/ml to 2 ng/ml.

Statistics:

Genotype and allelic frequencies between AD patients and controls were compared by Pearson's c² tests. Forward and backward unconditional logistic regression analyses were done for the simultaneous assessment of the influence of age, gender, APOE and CYP46 genotypes on the risk for developing AD. The estimate haplotype frequencies (EH) program was used to test for LD between SNPs. It computes the maximum-likelihood estimates for the haplotype frequencies assuming no association (H0) and allelic association (H1) and calculates the c² statistic as the two-fold difference between the log likelihoods (Terwilliger J D, et al., Handbook of Human Genetic Linkage, Baltimore: The Johns Hopkins University Press, 1994, pp.189-198). NFT stages and phases of b-amyloidosis between groups were compared with the U-test by Wilcoxon, Mann, and Whitney. For every comparison, statistical significance was assumed at p≦0.05.

TABLE 1

| | Hypothesis testing | | Hypothesis confirming | | | |
|---|---|---|---|---|---|---|
| | sample | | sample | | Combined sample | |
| | Control | | Control | | Control | AD |
| | subjects | AD patients | subjects | AD patients | subjects | patients |
| | (n = 76) | (n = 107) | (n = 172) | (n = 76) | (n = 248) | (n = 183) |
| CTP46 genotype | | | | | | |
| CT or CC | 41 (53.9%) | 42 (39.3%) | 98 (57.0%) | 32 (42.1%) | 139 (56.0%) | 74 (40.4%) |
| TT | 35 (46.1%) | 65 (60.7%) | 74 (43.0%) | 44 (57.9%) | 109 (44.0%) | 109 (59.6%) |
| Statistics | Pearson's χ² = 3.87 | | Pearson's χ² = 4.67 | | Pearson's χ² = 10.27 | |
| | p = 0.049 | | p = 0.031 | | p = 0.001 | |
| Allele | | | | | | |
| frequency | | | | | | |
| C | 0.309 | 0.220 | 0.340 | 0.250 | 0.331 | 0.232 |
| T | 0.691 | 0.780 | 0.660 | 0.750 | 0.669 | 0.768 |

| | | | |
|---|---|---|---|
| Statistics | Pearson's χ² = 3.74 | Pearson's χ² = 3.98 | Pearson's χ² = 9.92 |
| | p = 0.053 | p = 0.045 | p = 0.002 |

TABLE 2

| | | | |
|---|---|---|---|
| | Significance | Adjusted Odds | |
| Independent variable | (p) | ratio | 95% CI |

Hypothesis testing sample (107 AD patients, 76 control subjects)

| | | | |
|---|---|---|---|
| APOE*4 allele | 0.001 | 3.30 | 1.68-6.47 |
| CYP46*TT genotype | 0.044 | 1.90 | 1.02-3.55 |
| Age | 0.86 | — | — |
| Sex | 0.60 | — | — |

Hypothesis confirming sample (76 AD patients, 172 control subjects)

| | | | |
|---|---|---|---|
| APOE*4 allele | <0.0001 | 6.25 | 3.24-12.07 |
| CYP46*TT genotype | 0.008 | 2.42 | 1.26-4.68 |
| Age | 0.13 | — | — |
| Sex | 0.12 | — | — |

Combined sample (183 AD patients, 248 control subjects)

| | | | |
|---|---|---|---|
| APOE*4 allele | <0.00001 | 4.06 | 2.61-6.30 |
| CYP46*TT genotype | 0.0004 | 2.19 | 1.42-3.37 |
| Age | 0.97 | — | — |
| Sex | 0.08 | — | — |

TABLE 3

| | Odds ratio | 95% confidence inter- | |
|---|---|---|---|
| Comparison groups | (OR) | val (95% CI) | P value |
| APOE*4− | 1 | — | — |
| CYP46*TT− | | | |
| (reference group) | | | |
| APOE*4− | 2.03 | 1.13-3.67 | 0.012 |
| CYP46*TT+ | | | |
| APOE*4+ | 3.70 | 1.92-7.16 | 0.00002 |
| CYP46*TT− | | | |
| APOE*4+ | 9.07 | 4.37-19.07 | 2.2 * 10⁻¹⁰ |
| CYP46*TT+ | | | |

1. A method for diagnosing or prognosticating Alzheimer's disease in a subject, or determining the propensity or predisposition of a subject to develop Alzheimer's disease, which comprises detecting in a sample obtained from said subject the presence or absence of a variation in the CYP46 gene, wherein the presence of a variation in the CYP46 gene in said subject indicates a diagnosis or prognosis of Alzheimer's disease, or an increased propensity or predisposition to develop Alzheimer's disease as compared to a subject who does not carry a variation in said gene.

2. The method of claim 1, wherein said variation in the CYP46 gene is a single nucleotide polymorphism at a position 151 bp 5′ of exon 3 (single nucleotide polymorphism identification number: rs754203).

3. The method of claim 1, wherein said variation is a C to T transition.

4. The method of claim 1, wherein said subject is homozygous in respect to said variation.

5. The method of claim 1, further comprising detecting in a sample from said subject the presence of an apolipoprotein E4 allele.

6. The method of claim 5, wherein the presence of an apolipoprotein E4 allele in said subject indicates a diagnosis or prognosis of Alzheimer's disease, or a further increased propensity or predisposition to develop Alzheimer's disease.

7. The method according to claim 1, wherein said sample comprises DNA obtained from body fluids or tissues, preferably from blood.

8. A method for diagnosing or prognosticating Alzheimer's disease in a subject, or determining the propensity or predisposition of a subject to develop Alzheimer's disease, comprising:
determining a level, or an activity, or both said level and said activity, of at least one substance which is selected from the group consisting of a transcription product of the CYP46 gene or a translation product of the CYP46 gene in a sample from said subject; and comparing said level, or said activity, or both said level and said activity, of at least one of said substances to a reference value representing a known disease or health status, thereby diagnosing or prognosticating Alzheimer's disease in said subject, or determining the propensity or predisposition of said subject to develop Alzheimer's disease.

9. A method of monitoring the progression of Alzheimer's disease in a subject, comprising:
determining a level, or an activity, or both said level and said activity, of at least one substance which is selected from the group consisting of a transcription product of the CYP46 gene or a translation product of the CYP46 gene in a sample from said subject; and comparing said level, or said activity, or both said level and said activity, of at least one of said substances to a reference value representing a known disease or health status, thereby monitoring the progression of Alzheimer's disease in said subject.

10. A method of evaluating a treatment for Alzheimer's disease, comprising:
determining a level, or an activity, or both said level and said activity, of at least one substance which is selected from the group consisting of a transcription product of the CYP46 gene or a translation product of the CYP46 gene in a sample obtained from a subject being treated for Alzheimer's disease; and comparing said level, or said activity, or both said level and said activity, of at least one of said substances to a reference value representing a known disease or health status, thereby evaluating said treatment for Alzheimer's disease.

11. The method according to claim 8, wherein said sample is taken from a body fluid, a tissue, or an organ, preferably from cerebrospinal fluid.

12. The method according to claim 8, wherein said reference value is that of a level, or an activity, or both said level and said activity, of a transcription product of the CYP46 gene or a translation product of the CYP46 gene in a sample from a subject not suffering from Alzheimer's disease.

13. The method according to claim 8 further comprising comparing a level, or an activity, or both said level and said activity, of a transcription product of the CYP46 gene or a translation product of the CYP46 gene in a series of samples taken from said subject over a period of time.

14. The method according to claim 13, wherein said subject receives a treatment prior to one or more sample gatherings.

15. The method of claim 14, wherein said level, or said activity, or both said level and said activity, in said samples is determined before and after said treatment of said subject.

16. A kit for diagnosing or prognosticating Alzheimer's disease in a subject, or determining the propensity or predisposition of a subject to develop Alzheimer's disease, said kit comprising:
(a) at least one reagent which is selected from the group consisting of (i) reagents that selectively detect a transcription product of the CYP46 gene, (ii) reagents that selectively detect a translation product of the CYP46 gene, (iii) reagents that selectively detect the presence or absence of a variation in the CYP46 gene; and
(b) instruction for diagnosing, or prognosticating Alzheimer's disease, or determining the propensity or predisposition of a subject to develop Alzheimer's disease by
detecting a level, or an activity, or both said level and said activity, of said transcription product and/or said translation product of the CYP46 gene, in a sample from said subject; and/or detecting the presence or absence of a variation in the CYP46 gene in a sample from said subject; and
diagnosing or prognosticating Alzheimer's disease, or determining the propensity or predisposition of said subject to develop Alzheimer's disease,
wherein a varied level, or activity, or both said level and said activity, of said transcription product and/or said translation product compared to a reference value representing a known health status; or a level, or activity, or both said level and said activity, of said transcription product and/or said translation product similar or equal to a reference value representing a known disease status;
or the presence of a variation in the CYP46 gene indicates a diagnosis or prognosis of Alzheimer's disease, or an increased propensity or predisposition of developing Alzheimer's disease.

17. The kit according to claim 16, wherein said variation in the CYP46 gene is a single nucleotide polymorphism at the position 151 bp 5′ of exon 3 (single nucleotide polymorphism identification number: rs754203).

18. The kit according to claim 16, wherein the variation is a C to T transition.

19. The kit according to claim 16, wherein said subject is homozygous in respect to said variation.

20. The kit according to claim 16, further comprising reagents that selectively detect the presence or absence of an apolipoprotein E4 allele.

21. The kit according to claim 20, wherein the presence of an apolipoprotein E4 allele indicates a diagnosis or prognosis of Alzheimer's disease, or a further increased propensity or predisposition of developing Alzheimer's disease.

22. The kit according to claim 16 for use in monitoring a progression of Alzheimer's disease in a subject.

23. The kit according to claim 16 for use in monitoring success or failure of therapeutic treatment of said subject.
